(19) United States
(12) Patent Application Publication
(10) Pub. No.: US 2026/0108634 A1
(43) Pub. Date: Apr. 23, 2026

(54) HIGH MOLECULAR WEIGHT MULTI-ARM POLYMERS AND MEDICAL HYDROGELS FORMED THEREFROM

(71) Applicant: Boston Scientific Scimed, Inc., Maple Grove, MN (US)

(72) Inventors: Yen-Hao Hsu, Shrewsbury, MA (US); Marc Plourde, Worcester, MA (US); Heather Cirka, Hudson, MA (US)

(73) Assignee: Boston Scientific Scimed, Inc., Maple Grove, MN (US)

(21) Appl. No.: 19/364,201

(22) Filed: Oct. 21, 2025

Related U.S. Application Data
(60) Provisional application No. 63/710,188, filed on Oct. 22, 2024.

(51) Int. Cl. A61K 49/04

(52) U.S. Cl. CPC A61K 49/0409

(57) ABSTRACT

In some aspects, the present disclosure pertains to a kit for forming a cross-linked hydrogel, the kit comprising a reactive high molecular weight multi-arm polymer comprising a core, a plurality of polymer segments having a first end that is covalently attached to the core and a second end comprising a moiety that comprises a reactive group, wherein the reactive high molecular weight radiopaque multi-arm polymer has a molecular weight of greater than 10,000 Daltons; and a multifunctional compound that comprises functional groups that are reactive with the reactive group of the reactive high molecular weight multi-arm polymer.

### CROSS-REFERENCE TO RELATED APPLICATIONS

This application claims the benefit of U.S. Provisional Patent Application Ser. No. 63/710,188 filed on Oct. 22, 2024, the disclosure of which is incorporated herein by reference.

### FIELD

The present disclosure relates to high molecular weight multi-arm polymers and hydrogels formed therefrom. Hydrogels are useful, for example, in various medical applications.

### BACKGROUND

Injectable hydrogels are an emerging class of materials having a variety of medical uses. As one specific example, injectable hydrogels have been used to create or maintain space between tissues, for instance, to reduce side effects of off-target radiation therapy. For example, the hydrogel creates a space between a rectum and a prostate of a patient, moving the rectum away from the treatment region. This can help to reduce radiation exposure to the rectum and/or provide other desirable benefits.

The use of a hydrogel-based perirectal spacer material in conjunction with prostate radiation therapy is illustrated schematically in FIGS. 1A and 1B. FIG. 1A illustrates a cross-section of the human male anatomy including the prostate 110 and rectal wall 112. When the prostate is treated using radiation therapy there is a higher dose region 114 adjacent to the prostate, which is subjected to high doses of radiation, becoming a lower dose region 116 as one proceeds further from the prostate 110. As illustrated in FIG. 1B, a spacing material 118 can be injected between the prostate 110 and the rectal wall 112, which can push the rectal wall from a higher dose region 114 to a lower dose region 116, thereby reducing injury to the rectal wall.

SpaceOAR® and SpaceOAR Vue® are hydrogels that rapidly form crosslinks in-vivo. They are based on multi-arm PEG polymers having a polyol residue core functionalized with succinimidyl glutarate (SG) activated ester end groups. The multi-arm PEG iodinated polymer of SpaceOAR Vue® has a molecular weight of 10,000 Daltons or less. Above a specific pH, the SG groups will rapidly react with a multifunctional crosslinker such as a trilysine acetate (a lysine trimer) crosslinker in-vivo to form a hydrogel. The hydrogel thus contains a residue of the multi-arm PEG having a polyol residue core (Core) functionalized with SG end groups and a residue of the trilysine acetate. For example, with reference to FIG. 2, a succinimidyl-ester-terminated multi-arm PEG polymer (210) (only one arm is illustrated) of SpaceOAR® which has a molecular weight of 15,000 Daltons reacts with a nucleophilic amine group of crosslinking agent in the form of trilysine acetate (220) (only one amine group is illustrated) to yield a crosslinked hydrogel product (230) with crosslinks (only one crosslink is illustrated). The crosslinked hydrogel product (e.g., a non-iodinated hydrogel of SpaceOAR®) breaks down in-vivo, for instance, over the course of ca. 6-9 months before being completely expelled. The breakdown occurs primarily through the hydrolysis of the ester linkages on the glutarate groups.

However, for various reasons alternative reactive multi-arm polymers (e.g., PEG based reactive multi-arm polymers) are desired for forming crosslinked hydrogels.

### SUMMARY

In an aspect, a kit for forming a cross-linked hydrogel is provided. The kit comprising: a reactive high molecular weight multi-arm polymer comprising a core, a plurality of polymer segments having a first end that is covalently attached to the core and a second end comprising a moiety that comprises a reactive group, wherein the reactive high molecular weight radiopaque multi-arm polymer has a molecular weight of greater than 10,000 Daltons; and a multifunctional compound that comprises functional groups that are reactive with the reactive group of the reactive high molecular weight multi-arm polymer.

In some aspects, which may be used in combination with any of the other aspects herein, wherein the high molecular weight multi-arm polymer has a molecular weight in a range from greater than 10,000 Daltons to about 40,000 Daltons.

In some aspects, which may be used in combination with any of the other aspects herein, wherein the high molecular weight multi-arm polymer has a molecular weight in a range from greater than 10,000 Daltons to about 15,000 Daltons.

In some aspects, which may be used in combination with any of the other aspects herein, wherein the reactive high molecular weight multi-arm polymer has 3 to 12 polymer segments, and wherein at least some of the polymer segments contains a quantity of monomer units that is greater than 1000 monomer units.

In some aspects, which may be used in combination with any of the other aspects herein, wherein the reactive groups comprise electrophilic groups.

In some aspects, which may be used in combination with any of the other aspects herein, wherein the electrophilic groups comprise succinimide ester groups, propiolate groups, acrylate groups, ketone groups, aldehyde groups, or any combination thereof.

In some aspects, which may be used in combination with any of the other aspects herein, wherein the reactive high molecular weight multi-arm polymer further comprises a reactive high molecular weight radiopaque multi-arm polymer with a first subset of the plurality of polymer segments comprising polymer segments where the second end comprises a radiopaque group and a second subset of the plurality of polymer segments comprising polymer segments where the second end comprises the reactive group.

In some aspects, which may be used in combination with any of the other aspects herein, wherein a ratio of the radiopaque groups to the reactive groups is in a range from about 10:90 to about 40:60.

In some aspects, which may be used in combination with any of the other aspects herein, wherein a ratio of the radiopaque groups to the reactive groups is 40:60.

In some aspects, which may be used in combination with any of the other aspects herein, wherein the radiopaque group is a residue of an iodinated aromatic ring-based carboxylic acid compound

In some aspects, which may be used in combination with any of the other aspects herein, the iodinated aromatic ring-based carboxylic acid compound is a highly iodinated benzoic acid containing compound having at least 4 iodine groups.

In some aspects, which may be used in combination with any of the other aspects herein, wherein the highly iodinated benzoic acid containing compound is selected from 2,3,4,5-tetraiodbenzoic acid, 2,3,5,6-tetraiodobenzoic acid, 2,3,4,5,6-pentaiodbenzoic acid, or any combination thereof.

In some aspects, which may be used in combination with any of the other aspects herein, wherein the multifunctional compound is a polyamine, and wherein the polyamine further comprises a cationic polymer.

In some aspects, which may be used in combination with any of the other aspects herein, wherein the reactive high molecular weight multi-arm polymer has a melting temperature above normothermia.

In some aspects, which may be used in combination with any of the other aspects herein, wherein the plurality of polymer segments comprises polyethylene glycol (PEG) segments.

In some aspects, a system for forming a radiopaque cross-linked hydrogel is provided. The system comprising: (a) a reactive high molecular weight multi-arm radiopaque polymer comprising a core, a plurality of polymer segments having a first end that is covalently attached to the core and a second end comprising a moiety that comprises a reactive group or radiopaque group, wherein the reactive high molecular weight radiopaque multi-arm polymer has a molecular weight of greater than 10,000 Daltons, and wherein the second end of a first subset of the plurality of polymer segments comprises the reactive group, and wherein the second end of the first subset of the plurality of polymer segments comprises the radiopaque group; and (b) a multifunctional amine that comprises amino groups that are reactive with the reactive group of the reactive high molecular weight radiopaque multi-arm polymer.

In some aspects, which may be used in combination with any of the other aspects herein, wherein: the polymer segments comprise polyethylene glycol segments; the reactive groups comprise succinimidyl glutarate (SG) end groups, propiolate groups, acrylate end groups, ketone groups, aldehyde groups, or any combination thereof, and the multifunctional amine comprises trilysine acetate.

In some aspects, a cross-linked reaction product is provided. The cross-linked reaction product is formed from: (a) a reactive high molecular weight multi-arm radiopaque polymer comprising a core, a plurality of polymer segments having a first end that is covalently attached to the core and a second end comprising a moiety that comprises a reactive group or radiopaque group, wherein the reactive high molecular weight radiopaque multi-arm polymer has a molecular weight of in a range from greater than 10,000 Daltons to about 25,000 Daltons and has a melting temperature above normothermia, and wherein the second end of a first subset of the plurality of polymer segments comprises the reactive group, and wherein the second end of the first subset of the plurality of polymer segments comprises the radiopaque group; and (b) a multifunctional amine that comprises amino groups that are reactive with the reactive group of the reactive high molecular weight radiopaque multi-arm polymer.

In some aspects, which may be used in combination with any of the other aspects herein, wherein: a ratio of the radiopaque groups to the reactive groups is tunable and is in a range from about 10:90 to about 35:65; and the radiopaque group is a residue of compound a highly iodinated benzoic acid containing compound having at least 4 iodine groups.

In some aspects, which may be used in combination with any of the other aspects herein, wherein crosslinked reaction product exhibits improved cross-linking kinetics, in vivo gel persistence, improved radiopacity, or any combination thereof.

The above and other aspects, embodiments, features and benefits of the present disclosure will be readily apparent from the following detailed description.

### BRIEF DESCRIPTION OF THE DRAWINGS

FIG. 1A illustrates a cross-section of the human male anatomy including the prostate and rectal wall.

FIG. 1B illustrates an example of a spacing material between the prostate and the rectal wall.

FIG. 2 schematically illustrates an example process for forming a prior art crosslinked hydrogel product.

FIG. 3 schematically illustrates an example process for forming a reactive high molecular weight radiopaque multi-arm polymer suitable to form a cross-linked hydrogel, in accordance with an embodiment of the present disclosure.

FIG. 4 schematically illustrates an example process for forming a reactive high molecular weight multi-arm radiopaque polymer suitable to form a cross-linked hydrogel, in accordance with an embodiment of the present disclosure.

FIG. 5 schematically illustrates an example process for forming a reactive high molecular weight multi-arm radiopaque polymer suitable to form a cross-linked hydrogel, in accordance with an embodiment of the present disclosure.

FIG. 6 schematically illustrates an example process for forming a crosslinked hydrogel from a reactive high molecular weight multi-arm radiopaque polymer, in accordance with an embodiment of the present disclosure.

FIG. 7 illustrates a delivery device, in accordance with an embodiment of the present disclosure.

FIG. 8 illustrates a delivery device, in accordance with another embodiment of the present disclosure.

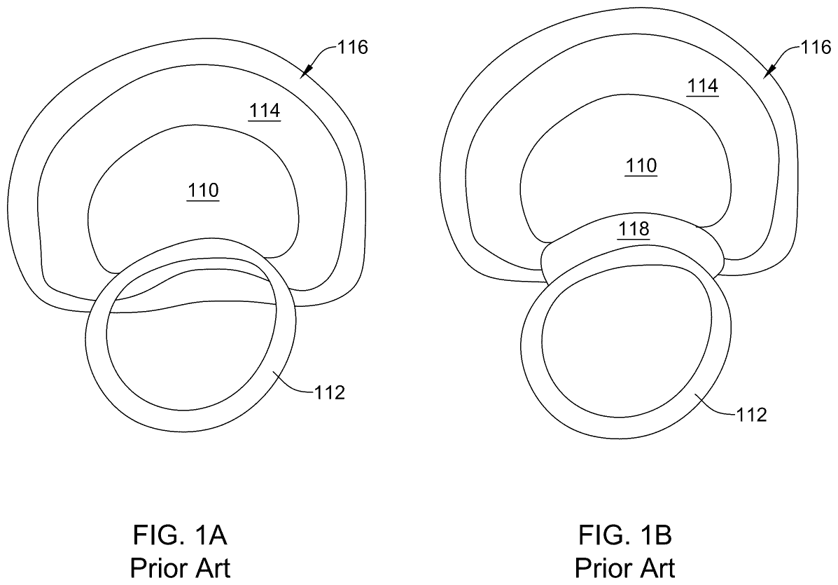

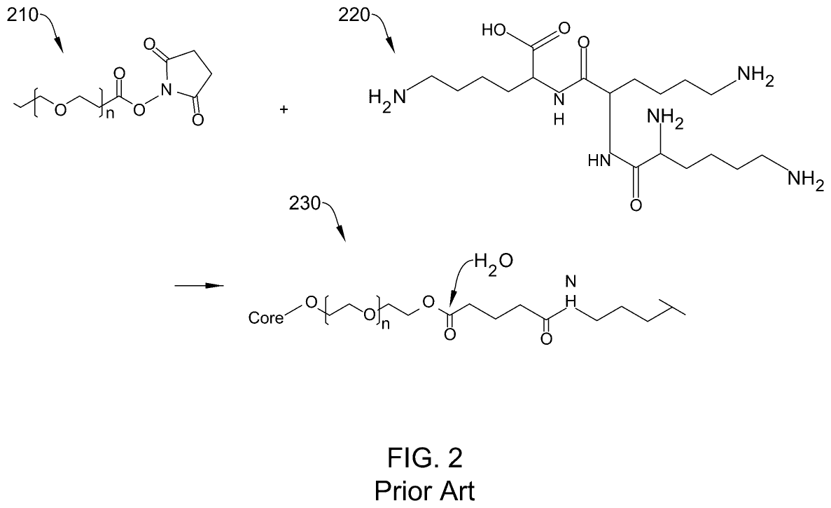

### DETAILED DESCRIPTION

The present disclosure provides kits and systems for forming a cross-linked medical hydrogel. The kits and systems herein can comprise a reactive high molecular weight multi-arm polymer comprising a core, a plurality of polymer segments (e.g., arms) having a first end that is covalently attached to the core and a second end comprising a moiety that comprises a reactive group, wherein the reactive high molecular weight radiopaque multi-arm polymer has a molecular weight of greater than 10,000 Daltons; and a multifunctional compound that comprises functional groups that are reactive with the reactive group of the multi-arm polymer, as described herein. For instance, the reactive high molecular weight multi-arm polymer can have a molecular weight in a range from greater than 10,000 Daltons to about 40,000 Daltons, as compared to typical iodinated multi-arm PEG polymers having a molecular weight of 10,000 Daltons or less. The reactive high molecular weight multi-arm polymers and hydrogels formed therefrom can exhibit improved properties such as improved solubility, improved melting temperatures, improved homogeneity, improved viscosity, and/or improved hydrophilicity. For example, the reactive high molecular weight multi-arm polymer and hydrogels formed therefrom can having a melting point above ambient temperature and/or above normothermia (e.g., a melting point at or above 40° C.), which is important for storage and transport. Alternatively or additionally, the high molecular weight multi-arm polymer and hydrogels that are formed therefrom can (despite the presence of additional monomers forming the higher molecular weight multi-arm polymers) still exhibit suitable or even enhanced gel persistence, curing kinetics, and radiopacity, as described herein. For instance, despite the increased molecular weight, the present disclosure aims to maintain curing kinetics similar to various commercialized products like SpaceOAR Classic.

In some embodiments, a quantity of arms (e.g., polymer segments) of the reactive high molecular weight multi-arm polymers can be varied. Varying the quantity of arms (while maintaining the high molecular weight) permits various properties (e.g., viscosity, melting temperature, moles of end groups, etc.) of the high molecular weight multi-arm polymer and the hydrogels formed therefrom to be tuned, as described herein, as described herein.

In some embodiments, the reactive high molecular weight multi-arm polymer can be a radiopaque high molecular weight multi-arm polymer. For instance, the radiopaque high molecular weight multi-arm polymer that can be functionalized with radiopaque groups formed from a highly iodinated benzoic acid compound (e.g., having at least four iodine substitutions). Thus, in such embodiments the radiopaque multi-arm polymers can exhibit suitable or increased radiopacity and/or permitting inclusion of a relatively high amount (e.g., greater than 60 mole percent) of reactive groups in the radiopaque high molecular weight multi-arm polymer, as compared to typical iodinated multi-arm PEG polymers (e.g., SpaceOAR Vue® with a molecular weight of 10,000 Daltons) which may contain fewer reactive groups (e.g., 40 or less mole percent reactive groups). That is, the presence of the highly iodinated benzoic acid derivative can be particularly beneficial in the context of the high molecular weight radiopaque multi-arm polymers herein, which due to the high molecular weight would otherwise tend exhibit reduced radiopacity (e.g., due the presence of an increased quantity of monomers therein).

The reactive high molecular weight multi-arm polymers herein (e.g., radiopaque high molecular weight multi-arm polymers) can have PEG based polymer segments and therefore are amenable to readily form hydrogels with various multifunctional compounds (e.g., crosslinkers) such as various amines (e.g., trilysine acetate (TLA)).

In some embodiments, the reactive groups of the reactive high molecular weight multi-arm polymers herein can include succinimidyl glutarate (SG) groups and/or other types of reactive end groups such as acrylate groups, as described herein. That is, in some embodiments a portion or all of the succinimidyl glutarate (SG) groups which may typically be present can be replaced with other types of reactive end groups such as acrylate groups (e.g., acrylate end groups, propiolate end groups) that are reactive with the functional groups having nucleophilicity (e.g., NH₂ groups, RNH groups that R can be any alkyl chain) the multifunctional compounds, described herein. In such embodiments, replacing some or all of the SG groups with another type of reactive end group yields enhanced tunability of various properties, such as those described herein, of the high molecular weight multi-arm polymer and the hydrogels formed therefrom. Michael-addition through Amino-ene reaction offers another crosslinking method while maintaining degradability with tunability of kinetics.

The above differences and improvements address several limitations of current hydrogels including cold chain issues, hydrophobicity concerns, and/or the ability to fine-tune various properties of the hydrogel for specific medical applications such as those in prostate cancer treatment.

Reactive high molecular weight multi-arm polymers herein have a molecular weight that is greater than 10,000 Daltons. For instance, the reactive high molecular weight multi-arm polymers herein can have a molecular weight in a range from greater than about 10,000 Daltons to about 40,000 Daltons. For example, the reactive high weight multi-arm polymers herein can have a molecular weight in a range having a lower value of about 11,000 Daltons, about 12,000 Daltons, about 14,000 Daltons, or about 15,000 Daltons and an upper value of about 17,000 Daltons, about 18,000 Daltons, about 19,000 Daltons, about 20,000 Daltons, about 21,000 Daltons, about 22,000 Daltons, about 23,000 Daltons, about 24,000 Daltons, about 25,000 Daltons, about 30,000, about 35,000, or about 40,000 Daltons. The reactive high weight multi-arm polymers herein can have a molecular weight of about 12,000 Daltons, about 13,000 Daltons, about 14,000 Daltons, about 15,000, about 16,000 Daltons, about 17,000 Daltons, about 18,000 Daltons, about 19,000 Daltons, about 20,000 Daltons, about 25,000 Daltons, about 30,000 Daltons, about 35,000 Daltons, or about 40,000 Daltons. For instance, the reactive high weight multi-arm polymers herein can have a molecular weight of about 15,000 Daltons.

The relatively high molecular weight of the reactive high molecular weight multi-arm polymers herein can be attributable as least in part to the presence of an increased quantity of monomers units (e.g., poly(C₁-C₆-alkylene oxide) segments such as poly(ethylene oxide) (PEO) (also referred to as polyethylene glycol or PEG) segments) in the reactive high molecular weight multi-arm polymers. For instance, in some embodiments, the reactive high molecular weight multi-arm polymers herein can be formed with the same quantity of arms (e.g. polymer segments) as a quantity of arms (e.g., 2 to 12 arms or higher than 12 arms) of traditional multi-arm polymers but can include a greater quantity of monomer units per arm, as detailed herein.

As mentioned, the relatively high molecular weight (e.g., greater than 10,000 Daltons) of the reactive high molecular weight multi-arm polymers herein can impart other various desirable properties (e.g., suitable or improved solubility, melting temperatures, homogeneity, viscosity, and/or hydrophilicity). For instance, the reactive high molecular weight multi-arm polymers herein can have a melting temperature above ambient or room temperature and/or above normothermia (e.g., which is desirably at least for the purposes of procurement, storage, handling). The reactive high molecular weight multi-arm polymers herein can have a melting temperature that is equal to or greater than about 38 degrees Celsius, about 39 degrees Celsius, or about 40 degrees Celsius or higher. For example, the reactive high molecular weight multi-arm polymers can have melting temperature equal to about 40 degrees Celsius or higher.

Reactive high molecular weight multi-arm polymers in accordance with the present disclosure include polymers that comprise a plurality of polymer arms linked to a core region. The reactive multi-arm polymers may have two, three, four, five, six, seven, eight, nine, ten, eleven, twelve, thirteen, fourteen, fifteen, sixteen, seventeen, eighteen, nineteen, twenty, twenty-five, thirty or more polymer arms.

As discussed in more detail below, in some embodiments, the core region comprises a residue of a multi-functional initiator, specifically, a residue of an polyol initiator having two, three, four, five, six, seven, eight, nine, ten, eleven, twelve, thirteen, fourteen, fifteen, sixteen, seventeen, eighteen, nineteen, twenty, twenty-five, thirty or more hydroxyl groups, with the number of polymer arms corresponding to the number of functional groups, specifically, the number of hydroxyl groups, in the initiator that is used to form the reactive high molecular multi-arm polymer.

The polymer arms of the reactive multi-arm polymers may each comprise a polymer segment linked to the core region and a terminal reactive moiety.

In some embodiments, a hydrolysable moiety that comprises a hydrolysable linkage as described above is provided between the reactive moiety and the polymer segment.

Reactive moieties may be selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, and reactive moieties that comprise acrylate groups, reactive moieties that comprises propiolate groups, and among others.

Electrophilic groups include cyclic imide ester groups, including succinimide ester groups,

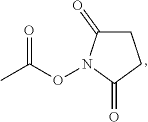

maleimide ester groups, glutarimide ester groups, diglycolimide ester groups, phthalimide ester groups, and bicyclo[2.2.1]hept-5-ene-2,3-dicarboxylic acid imide ester groups,

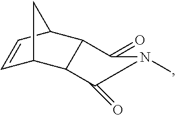

imidazole ester groups, imidazole carboxylate groups and benzotriazole ester groups, among other possibilities. Nucleophilic groups include primary amine groups, thiol groups and hydroxyl groups, among other possibilities. Diene containing groups include furan groups and tetrazine groups. Dienophile groups include norbornene groups and maleimide groups. Alkenyl-containing groups include vinyl groups, acryloyl groups, methacryloyl groups and strained alkene groups, for example, from cyclooct-4-en-1-yl groups,

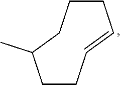

among other possibilities. Strained alkyne groups include (1R,8S,9s)-bicyclo[6.1.0]non-4-yn-9-yl groups,

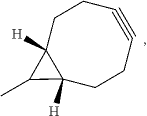

among other possibilities.

Polymer segments for the polymer arms can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(O-propiolactone) segments, poly(O-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Polymer segments for use in the higher molecular weight reactive multi-arm polymers of the present disclosure typically contain between 10 and 1000 monomer units or more. For instance, at least some of the polymer segments can contain a quantity of monomer units that is greater than 1000 monomer units, thus yielding the higher molecular weight reactive multi-arm polymers that have a molecular weight of greater than 10,000 Daltons, as described herein.

As previously noted, in various embodiments, the reactive multi-arm polymers of the present disclosure have two or more polymer arms that extend from a core region. In some of these embodiments, the core region comprises a residue of a polyol comprising two or more hydroxyl groups, which is used to form the polymer arms. In certain beneficial embodiments, the core region comprises a residue of a polyol that contains two, three, four, five, six, seven, eight, nine, ten, eleven, twelve, thirteen, fourteen, fifteen, sixteen, seventeen, eighteen, nineteen, twenty, twenty-five, thirty, or more hydroxyl groups.

In some embodiments, a quantity of the arms can be varied (e.g., to a quantity of arms that is in a range from 2 arm to 12 arms) while also maintaining a high molecular weight of the multi-arm polymer. For instance, a quantity of monomers can be reduced as a quantity of arms is increased and vice versa to ensure that the reactive multi-arm polymer has a target molecular weight (e.g., greater than 10,000 Daltons). As mentioned, tuning the quantity of arms can alter various properties of the high molecular weight multi-arm polymer and/or the hydrogels formed therefrom. For instance, increasing a quantity of arms can increase viscosity and melting temperature, while decreasing the quantity of arms can decrease a molar ratio of end groups that are present. Thus, the properties of the hydrogels formed from high molecular weight multi-arm polymers can be readily tuned, even when employed with typical multifunctional compounds (e.g., crosslinkers) such as TLA.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, polyhedral oligomeric silsesquioxanes (POSS), catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

In some embodiments, iodinated polyols may be employed to provide the resulting multi-arm polymer with radiopacity. In some of these embodiments, the iodinated polyols are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with the three or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others. Specific examples of iodinated polyols for use in the present disclosure include known iodinated contrast agents, whose biocompatibility has been demonstrated to be reasonably well tolerated, which include iopromide, iopamidol, iohexol, ioversol, and iodixanol.

In particular embodiments where an ester is selected as a hydrolysable linkage, terminal hydroxyl groups of the polymer segments maybe reacted with acyclic anhydride compound (e.g., glutaric anhydride, succinic anhydride, malonic anhydride, adipic anhydride, diglycolic anhydride, 1,3-acetonedicarboxylic acid anhydride, etc.) to form an acid-end-capped polymer segment such as a glutaric-acid-end-capped segment, a succinic-acid-end-capped segment, a malonic-acid-end-capped segment, an adipic-acid-end-capped segment, a diglycolic-acid-end-capped segment, a 1,3-acetonedicarboxylic-acid-end-capped segment, and so forth.

The preceding cyclic anhydrides, among others, may be reacted with a hydroxy-terminated multi-arm hydrophilic precursor polymer under basic conditions to form a carboxylic-acid-terminated precursor polymer comprising a carboxylic acid end group that is linked to a polymer segment through a hydrolysable ester group.

A reactive moiety may then be linked to the carboxylic-acid-terminated precursor polymer. For example, in particular embodiments where a cyclic imide ester group is employed as a reactive group, an N-hydroxy cyclic imide compound (e.g., N-hydroxysuccinimide, N-hydroxymaleimide, N-hydroxyglutarimide, N-hydroxyphthalimide, N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl\grave{ }propyl)carbodiimide (EDC), N-hydroxybenzotriazole (HOBt), BOP reagent, and/or another coupling agent) to form a reactive cyclic imide ester group (e.g., a succinimide ester group, a maleimide ester group, a glutarimide ester group, a phthalimide ester group, a bicyclo[2.2.1]hept-5-ene-2,3-dicarboxylic acid imide ester group, etc.) that is linked to a polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, succinimidyl diglycolate groups, and succinimidyl 1,3-acetonedicarboxylate groups (1,3-acetonedicarboxylate groups may also be referred to herein as 3-oxopentanedioate groups), among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, bicyclo[2.2.1]hept-5-ene-2,3-dicarboxylic acid imidyl diglycolate groups, and bicyclo[2.2.1]hept-5-ene-2,3-dicarboxylic acid imidyl 1,3-acetonedicarboxylate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, maleimidyl diglycolate groups, and maleimidyl 1,3-acetonedicarboxylate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, and glutarimidyl 1,3-acetonedicarboxylate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, phthalimidyl diglycolate groups, and phthalimidyl 1,3-acetonedicarboxylate groups, among others.

Using various suitable chemistries, hydrolysable moieties that comprise hydrolysable linkages other than the ester linkages described above can also be inserted into the arms of reactive multi-arm polymers. For example, such hydrolysable moieties may be inserted between a polymer segment described herein and a reactive moiety as described herein. Such hydrolysable linkages may be selected, for example, from an ester linkage other than one of those described above, a carbonate linkage, an acid anhydride linkage, an imide linkage, a ketal linkage, a carbamate linkage, an organophosphate ester linkage, a silane linkage, an amide linkage, a hydrozonium linkage, an acylhydrozone linkage, an oxime linkage, and an amidohydrozone linkage.

Furthermore, as noted above, such hydrolysable moieties may further include activating groups positioned proximate to the hydrolysable linkages, which increase the rate of hydrolysis of the hydrolysable linkages. Particular examples of activating groups include, for example, urea linkages, urea pendant groups, thiourea linkages, thiourea pendant groups, amine linkages, amine pendant groups, alcohol pendant groups, silicon-containing linkages silicon-containing pendant groups, boron-containing linkages, boron-containing pendant groups, phosphonate linkages, phosphonate pendant groups, and sulfonate pendant groups, as described above.

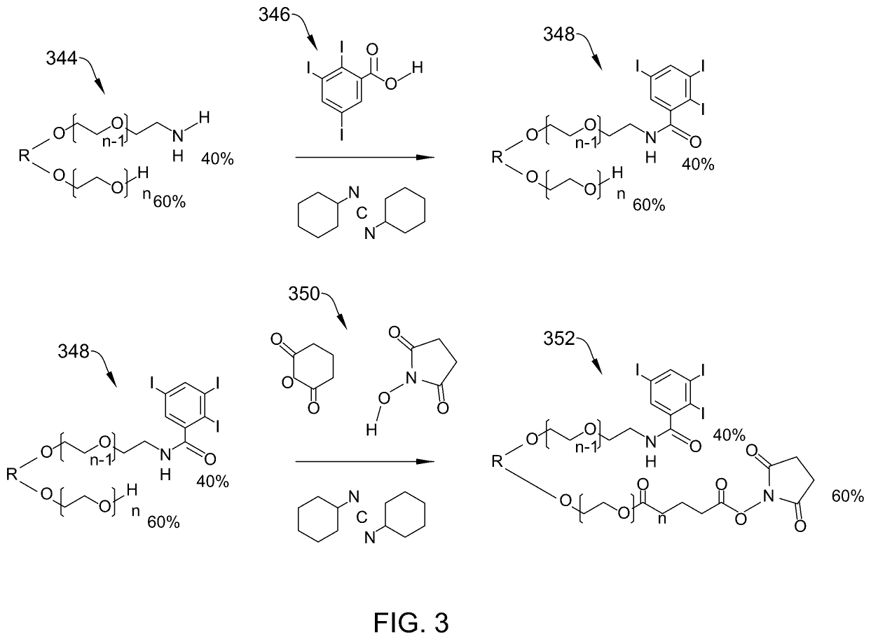

FIG. 3 schematically illustrates an example process for forming a reactive high molecular weight radiopaque multi-arm polymer suitable to form a cross-linked hydrogel. The process in FIG. 3 is analogous to the process used to form traditional reactive multi-arm radiopaque polymers (e.g., those employed in SpaceOAR Vue®), with the change that a reactive high molecular weight multi-arm polymer 344 is employed as the starting material. The reactive high molecular weight multi-arm polymer 344 can be manifested as an 8-arm polymer (e.g., only two arms are illustrated in FIG. 3) with PEG linkages and a molecular weight of greater than 10,000 Daltons. The multi-arm polymer 344 can be reacted with various components (e.g., can undergo DIC or DCC coupling with a suitable coupling agent such as a carbodiimide coupling agent such as N,N′-dicyclohexylcarbodiimide (DCC)) to yield the reactive high molecular weight radiopaque multi-arm polymer 352. For instance, end groups (e.g., amino groups) of the polymer 344 can be functionalized (as illustrated at 346) to obtain a polymer with radiopaque end groups (e.g., 2,3,5-triiodobenzamide (TIB) end groups, as illustrated at 348). That is, the end groups of the polymer 344 can be reacted with an iodinated aromatic ring-based carboxylic acid compound to form radiopaque groups that are a residue of the iodinated aromatic ring-based carboxylic acid compound (e.g., are functionalized with 2,3,5-triiiodobenzamide (TIB) groups, as illustrated in FIG. 3).

The iodinated aromatic ring-based carboxylic acid compound can comprise a polyiodinated aromatic moiety having a plurality (two, three, four, five, six, seven, eight, nine, ten or more) iodine groups. For example, the polyiodinated aromatic moiety may comprise a monocyclic or multicyclic aromatic structure that is substituted with (a) a plurality of iodine groups (e.g., two, three, four, five, six, seven, eight, nine, ten or more iodine groups) and (b) one or a plurality of hydrophilic functional groups (e.g., one, two, three, four, five, six or more hydrophilic functional groups). The monocyclic or multicyclic aromatic structure may be selected, for example, from monocyclic aromatic structures such as those based on benzene and multicyclic aromatic structures such as those based on naphthalene (two aromatic rings) and/or anthracene (three aromatic rings), among others. The one or the plurality of hydrophilic functional groups may comprise, for example, hydroxyalkyl groups such as C1-C4-hydroxyalkyl groups (e.g., C1-C4-monohydroxyalkyl groups, C1-C4-dihydroxyalkyl groups, C1-C4-trihydroxyalkyl groups, C1-C4-tetrahydroxyalkyl groups, etc.), among others. The hydroxyalkyl groups may be linked to the monocyclic or multicyclic aromatic structures directly or through any suitable linking moiety, which may be selected, for example, from amide groups, amine groups, ether groups, ester groups, or carbonate groups, among others. In some embodiments, the iodinated aromatic ring-based carboxylic acid compound can be a highly iodinated benzoic acid containing compound having at least 4 iodine groups. Examples of suitable highly iodinated benzoic acid derivatives include 2,3,4,5-tetraiodbenzoic acid, 2,3,5,6-tetraiodobenzoic acid, 2,3,4,5,6-pentaiodbenzoic acid, or any combination thereof.

The remaining end groups of the polymer 344 can be functionalized (e.g., as illustrated at 350, for instance with an N-hydroxy cyclic imide compound (e.g., N-hydroxysuccinimide, N-hydroxymaleimide, N-hydroxyglutarimide, N-hydroxyphthalimide, N-hydroxy-5-norbornene-2,3-dicarboxylic acid imide, also known as N-hydroxybicyclo[2.2.1]hept-5-ene-2,3-dicarboxylic acid imide (HONB), etc.) which may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl\grave{ }propyl)carbodiimide (EDC), N-hydroxybenzotriazole (HOBt), BOP reagent, and/or another coupling agent) to form a reactive high molecular weight radiopaque polymer 352 having reactive end groups (e.g., succinimide groups) and the radiopaque end groups (2,3,5-triiiodobenzamide (TIB) groups).

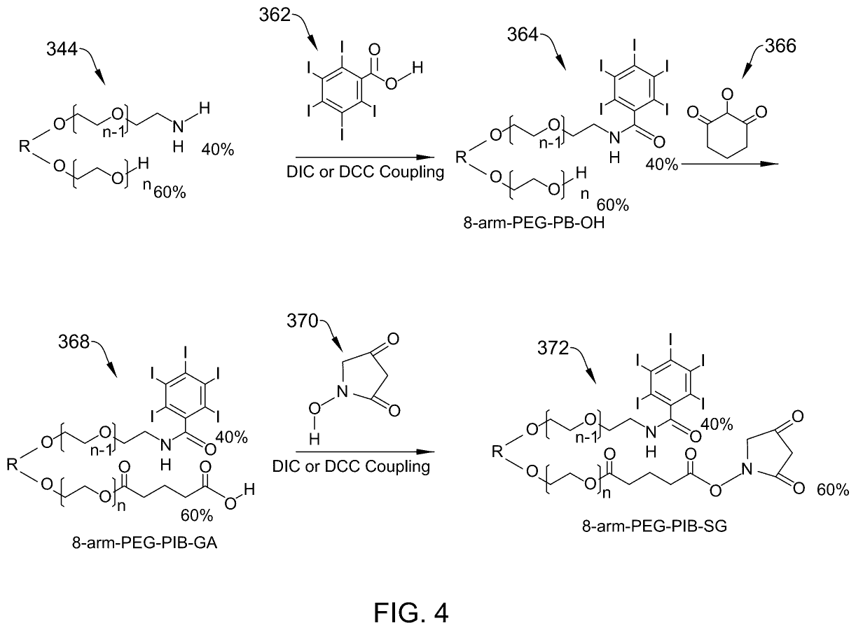

FIG. 4 schematically illustrates another example process for forming a reactive high molecular weight radiopaque multi-arm polymer 372. The process in FIG. 4 is analogous to the process in FIG. 3, with the change that a highly iodinated benzoic acid containing compound (e.g., 2,3,4,5,6-pentaiodbenzoic acid) having at least 4 iodine groups is used to form the radiopaque end groups of the reactive high molecular weight radiopaque multi-arm polymer 372. For instance, the end groups of the reactive high molecular weight multi-arm polymer 344 can be functionalized with 2,3,4,5,6-pentaiodbenzoic acid (as illustrated at 362) to obtain a polymer with radiopaque end groups (e.g., 2,3,4,5,6-pentaiodobenzamide end groups, as illustrated at 364). Employing the highly iodinated benzoic acid containing compound can account for the increased amount of monomers in the high molecular weight multi-arm polymers and thereby ensure that sufficient radiopacity of the hydrogels formed with the high molecular weight radiopaque multi-arm polymers is maintained (e.g., a molar concentration of the iodine groups remains the same or similar to a molar concentration of the iodine groups typically employed in the existing SpacerOAR Vue® hydrogels formed with multi-arm polymers having a molecular weight of 10,000 Daltons).

As illustrated in FIGS. 3-4, a ratio of the radiopaque groups to the reactive groups in the multi-arm polymers 348, 366 can be 40:60. However, in some embodiments a ratio of the radiopaque groups to the reactive groups can be varied. For instance, a ratio of the radiopaque groups to the reactive groups can be varied within in a range from about 10:90 to about 40:60. For example, the ratio of the radiopaque groups to the reactive groups can be 10:90, 15:85, 20:80, 25:75, 30:70, 35:65, or 40:60, among other possible ratios. Varying the ratio of the radiopaque groups to the reactive groups can permit various properties (e.g., radiopacity, cross-linking kinetics, solubility, hydrophilicity, etc.) of the high molecular weight radiopaque multi-arm polymers and the hydrogels formed therefrom to be tuned. For instance, decreasing the ratio of the radiopaque groups to the reactive groups can result in improvements in crosslinking kinetics (e.g., faster gelation times of the hydrogels) of the high molecular weight radiopaque multi-arm polymers. In some embodiments, the decreased ratio (e.g., a 35:65 ratio) of the radiopaque groups to the reactive groups can be employed in conjunction with a highly iodinated benzoic acid containing compound having at least 4 iodine groups thereby yielding improvements in crosslinking kinetics, and yet maintaining or improving radiopacity.

As illustrated in FIGS. 3-4, each of the reactive groups can be succinimidyl glutarate (SG) end groups. However, in some embodiments at least a portion of the reactive end groups can be formed of other types of reactive end groups that are suitable for hydrogel formation (e.g., via reaction with TLA). Varying a type of the reactive end groups (e.g., other than succinimidyl-ester-terminated end groups) can impart another degree of tunability to the reactive high molecular weight radiopaque multi-arm polymer and hydrogels formed therefrom.

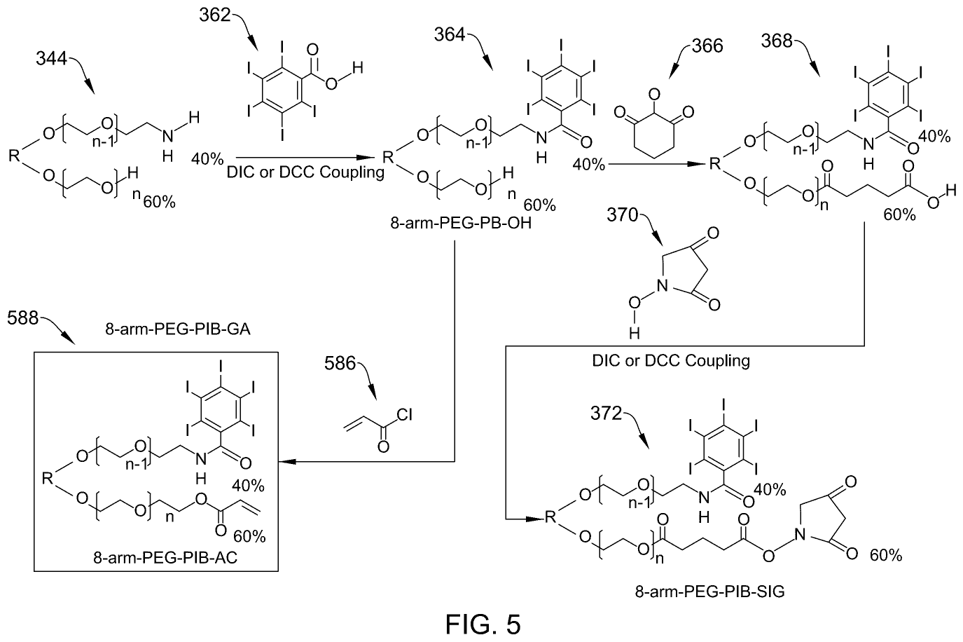

FIG. 5 schematically illustrates an example process for forming a reactive high molecular weight multi-arm radiopaque polymer suitable to form a cross-linked hydrogel. As illustrated in FIG. 5, a reactive high molecular weight radiopaque multi-arm polymer 352 having a succinimidyl-ester-terminated end groups and radiopaque end groups (iodine groups) can be formed from a reactive high molecular weight polymer 344 via the same or similar process as described with respect to FIG. 4 (e.g., as described with respect to elements 362, 364, 366, 368, and 370).

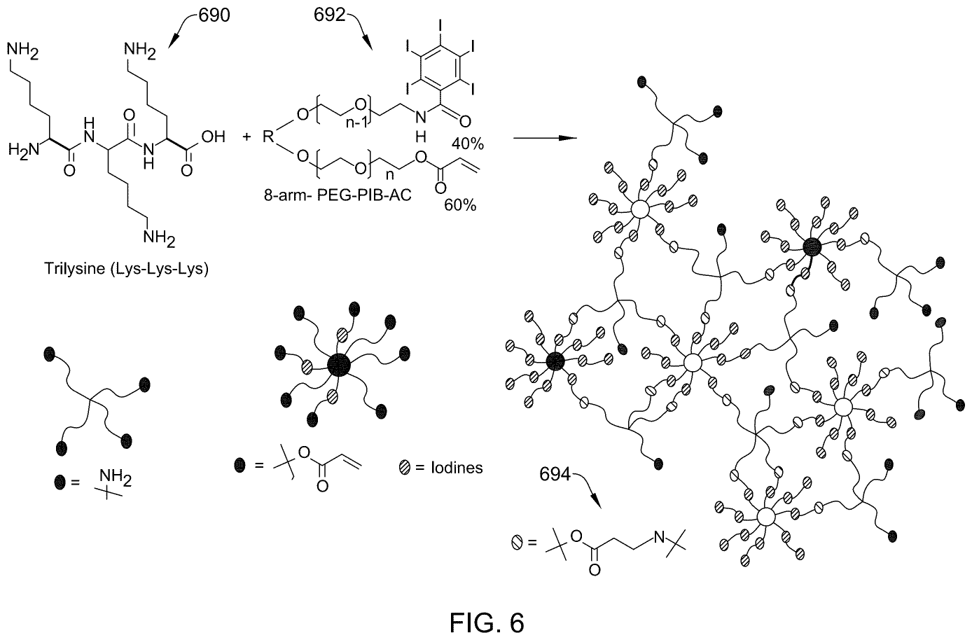

However, at least a portion or all of the reactive groups of the one or more arms of the reactive high molecular weight polymer 344 can be functionalized with a different type of reactive end group. For instance, the iodinated polymer 364 can be reacted with acryloyl chloride (as illustrated at 586) to yield the reactive high molecular weight radiopaque multi-arm polymer 588 having acrylate end groups. It is noted that the reactive high molecular weight radiopaque multi-arm polymer 588 is suitable to form a crosslinked product (e.g., a degradable radiopaque hydrogel) via Amino-ene Michael addition, for instance, as described with respect to FIG. 6 herein.

In the present disclosure, crosslinked hydrogels products are formed from a multi-arm polymer comprising a core, a plurality of segments having a first end that is covalently attached to the core and a second end comprising a moiety that comprises a reactive group. Examples of suitable multi-arm polymers (e.g., PEG polymers), are described herein. The multi-arm polymer is reacted with a multifunctional compound that comprises functional groups that are reactive with the reactive group of the multi-arm polymer.

In various embodiments, the multifunctional compound (e.g., for use in the compositions of the above systems and devices), and for use in forming the above crosslinked products, may be a polyamine. In general, polyamines suitable for use in the present disclosure include, for example, small molecule polyamines (e.g., containing at least two amine groups, for instance, from 3 to 20 amine groups in certain embodiments), comb polymers having amine side groups, and branched polymers having amine end groups, including dendritic polymers having amine end groups.

Particular examples of multifunctional amines which may be used as the multifunctional compound include trilysine, ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, among others. For instance, the multifunctional compound can be manifested as trilysine, in various embodiments.

FIG. 6 schematically illustrates an example process for forming a crosslinked hydrogel from a reactive high molecular weight multi-arm radiopaque polymer. The process of FIG. 6 can be employed with the systems and kits herein to form crosslinked hydrogels, either in vivo or ex vivo. For example, as shown schematically in FIG. 6, a composition comprising a reactive high molecular weight multi-arm polymer 692 like that described herein, which comprises a core region and a plurality of hydrophilic polymer arms having reactive end groups (where R is a core region, such as a polyol residue, and n ranges, for example, from 15 to 125) can be crosslinked with a composition comprising a multifunctional compound 690 like that described herein, which comprises amino groups that are reactive with the reactive groups of the reactive multi-arm polymer 692, to form a crosslinked product 694, which may be in the form of a hydrogel when hydrated.

In some aspects of the present disclosure, a system is provided that comprises: (a) a first composition comprising reactive high molecular weight multi-arm polymer (i.e., the multi-arm polymer), as described herein and (b) a second composition that comprises a multifunctional compound, as described herein, wherein the system is configured to deliver the reactive high molecular weight multi-arm polymer and the multifunctional compound under conditions such that covalent crosslinks are formed between the reactive high molecular weight multi-arm polymer and the multifunctional compound.

The first composition may be a first fluid composition comprising the multi-arm polymer or may be a first dry composition that comprises the multi-arm polymer, to which a suitable fluid such as water for injection, saline, etc. can be added to form the first fluid composition. In addition to the multi-arm polymer, the first composition may further comprise additional agents, including therapeutic agents, imaging agents, colorants, tonicity adjusting agents, suspension agents, wetting agents, and pH adjusting agents as described below.

The second composition may be a second fluid composition comprising the multifunctional compound or may be a second dry composition that comprises the multifunctional compound to which a suitable fluid such as water for injection, saline, etc. can be added to form a second fluid composition. In addition to the multifunctional compound, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the multi-arm polymer with a second fluid comprising the multifunctional compound. Upon mixing the first and second fluid compositions, the multifunctional compound crosslinks with the multi-arm polymer, forming a crosslinked product. The first and second fluid compositions may be combined form crosslinked hydrogels, either in vivo or ex vivo.

In some embodiments, the multifunctional compound is initially combined with the multi-arm polymer under conditions where crosslinking between the multi-arm polymer and the multifunctional compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the multifunctional compound and the multi-arm polymer, thereby forming a crosslinked product.

In some embodiments, the system comprises (a) a first composition, as described hereinabove, (b) a second composition, as described hereinabove, and, optionally, (c) a third composition, specifically, an accelerant composition, that contains an accelerant that is configured to accelerate a crosslinking reaction between the first composition and the second composition.

The second composition may be a second fluid composition comprising the multifunctional compound that is buffered to an acidic pH or a second dry composition that comprises the multifunctional compound to which a suitable fluid such as water for injection, saline, an acidic buffer solution, etc. can be added to form a second fluid composition comprising the multifunctional compound that is buffered to an acidic pH. In some embodiments, for example, the acidic buffering composition may comprise monobasic sodium phosphate, among other possibilities. The second fluid composition comprising the multifunctional compound may have a pH ranging, for example, from about 3 to about 5.

The first composition may be a first fluid composition comprising the multi-arm polymer or a first dry composition that comprises the multi-arm polymer from which a fluid composition is formed, for example, by the addition of a suitable fluid such as water for injection, saline, or the second fluid composition comprising the multifunctional compound that is buffered to an acidic pH.

In a particular embodiment, the second composition is a second fluid composition comprising the multifunctional compound that is buffered to an acidic pH and the first composition comprises a dry composition that comprises the multi-arm polymer. The second composition may then be mixed with the first composition to provide a prepared fluid composition that is buffered to an acidic pH and comprises the multifunctional compound and the multi-arm polymer. In a particular example, a syringe may be provided that contains the second fluid composition comprising the multifunctional compound that is buffered to an acidic pH, and a vial may be provided that comprises the dry composition (e.g., a powder) that comprises the multi-arm polymer. The syringe may then be used to inject the second fluid composition into the vial containing the reactive polymer to form a prepared fluid composition that is buffered to an acidic pH and contains the multifunctional compound and the multi-arm polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the multifunctional compound and the multi-arm polymer as described above, and a fluid accelerant composition that is buffered to basic pH as described above, may be combined form crosslinked hydrogels, either in vivo or ex vivo.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering the first and second compositions, as described herein, within a subject.

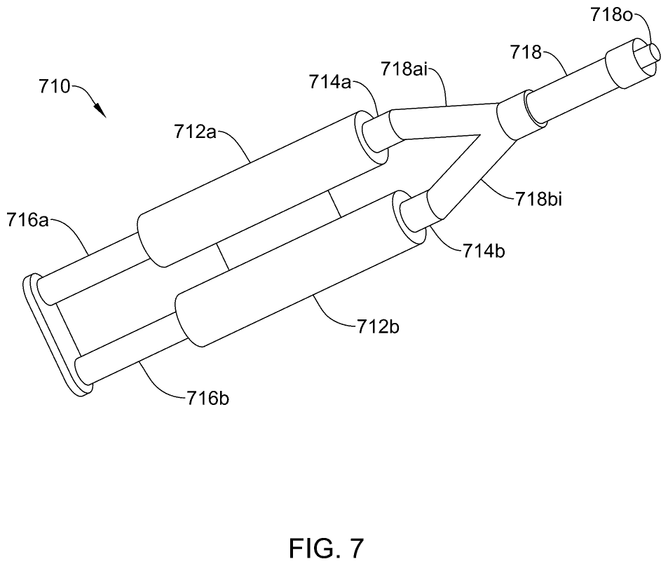

In particular embodiments, and with reference to FIG. 7, the system may include a delivery device 710 that comprises a double-barrel syringe, which includes a first barrel 712a having a first barrel outlet 714a, which first barrel contains the first composition, a first plunger 716a that is movable in the first barrel 712a, a second barrel 712b having a second barrel outlet 714b, which second barrel 712b contains the second composition, and a second plunger 716b that is movable in the second barrel 712b. In some embodiments, the device 710 may further comprise a mixing section 718 having a first mixing section inlet 718ai in fluid communication with the first barrel outlet 714a, a second mixing section inlet 718bi in fluid communication with the second barrel outlet, and a mixing section outlet 718o.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

In some embodiments, after administration the compositions of the present disclosure can be imaged using a suitable imaging technique. Typically, the imaging techniques is an x-ray-based imaging technique, such as computerized tomography or x-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may vary widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel composition to a subject. Such systems may include one or more of the following: a syringe barrel, which may or may not contain a crosslinked hydrogel composition as described herein; a vial, which may or may not contain a crosslinked hydrogel composition as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel composition may be provided in dry form (e.g., powder form) or in a form that is ready for injection, such as an injectable hydrogel form (e.g., a suspension of crosslinked hydrogel particles).

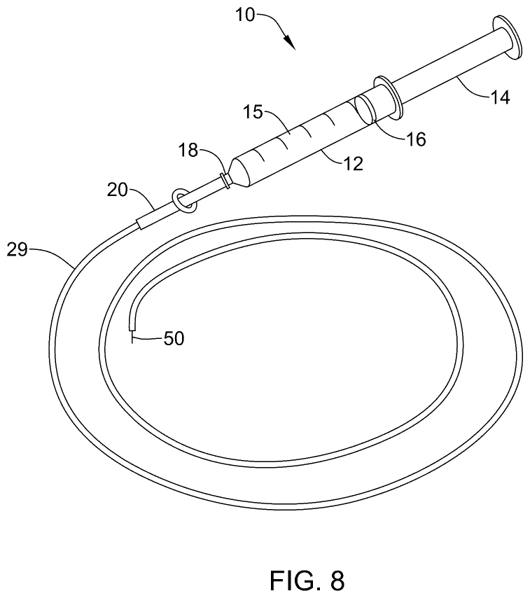

FIG. 8 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50. The crosslinked hydrogel compositions described herein can be used for a number of purposes including those described herein. For instance, crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

What is claimed is:

1. A kit for forming a cross-linked hydrogel, the kit comprising:
a reactive high molecular weight multi-arm polymer comprising a core, a plurality of polymer segments having a first end that is covalently attached to the core and a second end comprising a moiety that comprises a reactive group, wherein the reactive high molecular weight radiopaque multi-arm polymer has a molecular weight of greater than 10,000 Daltons; and
a multifunctional compound that comprises functional groups that are reactive with the reactive group of the reactive high molecular weight multi-arm polymer.

2. The kit of claim 1, wherein the high molecular weight multi-arm polymer has a molecular weight in a range from greater than 10,000 Daltons to about 40,000 Daltons.

3. The kit of claim 1, wherein the high molecular weight multi-arm polymer has a molecular weight in a range from greater than 10,000 Daltons to about 15,000 Daltons.

4. The kit of claim 1, wherein the reactive high molecular weight multi-arm polymer has 3 to 12 polymer segments, and wherein at least some of the polymer segments contains a quantity of monomer units that is greater than 1000 monomer units.

5. The kit of claim 4, wherein the reactive groups comprise electrophilic groups.

6. The kit of claim 5, wherein the electrophilic groups comprise succinimide ester groups, propiolate groups, acrylate groups, ketone groups, aldehyde groups, or any combination thereof.

7. The kit of claim 1, wherein the reactive high molecular weight multi-arm polymer further comprises a reactive high molecular weight radiopaque multi-arm polymer with a first subset of the plurality of polymer segments comprising polymer segments where the second end comprises a radiopaque group and a second subset of the plurality of polymer segments comprising polymer segments where the second end comprises the reactive group.

8. The kit of claim 7, wherein a ratio of the radiopaque groups to the reactive groups is in a range from about 10:90 to about 40:60.

9. The kit of claim 7, wherein a ratio of the radiopaque groups to the reactive groups is 40:60.

10. The kit of claim 7, wherein the radiopaque group is a residue of an iodinated aromatic ring-based carboxylic acid compound.

11. The kit of claim 10, wherein the iodinated aromatic ring-based carboxylic acid compound is a highly iodinated benzoic acid containing compound having at least 4 iodine groups.

12. The kit of claim 11, wherein the highly iodinated benzoic acid containing compound is selected from 2,3,4,5-tetraiodbenzoic acid, 2,3,5,6-tetraiodobenzoic acid, 2,3,4,5,6-pentaiodbenzoic acid, or any combination thereof.

13. The kit of claim 1, wherein the multifunctional compound is a polyamine, and wherein the polyamine further comprises a cationic polymer.

14. The kit of claim 1, wherein the reactive high molecular weight multi-arm polymer has a melting temperature above normothermia.

15. The kit of claim 14, wherein the plurality of polymer segments comprises polyethylene glycol (PEG) segments.

16. A system for forming a radiopaque cross-linked hydrogel, the system comprising:
(a) a reactive high molecular weight multi-arm radiopaque polymer comprising a core, a plurality of polymer segments having a first end that is covalently attached to the core and a second end comprising a moiety that comprises a reactive group or radiopaque group, wherein the reactive high molecular weight radiopaque multi-arm polymer has a molecular weight of greater than 10,000 Daltons, and wherein the second end of a first subset of the plurality of polymer segments comprises the reactive group, and wherein the second end of the first subset of the plurality of polymer segments comprises the radiopaque group; and
(b) a multifunctional amine that comprises amino groups that are reactive with the reactive group of the reactive high molecular weight radiopaque multi-arm polymer.

17. The system of claim 16, wherein:
the polymer segments comprise polyethylene glycol segments;
the reactive groups comprise succinimidyl glutarate (SG) end groups, propiolate groups, acrylate end groups, ketone groups, aldehyde groups, or any combination thereof, and
the multifunctional amine comprises trilysine acetate.

18. A cross-linked reaction product of:
(a) a reactive high molecular weight multi-arm radiopaque polymer comprising a core, a plurality of polymer segments having a first end that is covalently attached to the core and a second end comprising a moiety that comprises a reactive group or radiopaque group, wherein the reactive high molecular weight radiopaque multi-arm polymer has a molecular weight of in a range from greater than 10,000 Daltons to about 25,000 Daltons and has a melting temperature above normothermia, and wherein the second end of a first subset of the plurality of polymer segments comprises the reactive group, and wherein the second end of the first subset of the plurality of polymer segments comprises the radiopaque group; and
(b) a multifunctional amine that comprises amino groups that are reactive with the reactive group of the reactive high molecular weight radiopaque multi-arm polymer.

19. The cross-linked reaction product of claim 18, wherein:
a ratio of the radiopaque groups to the reactive groups is tunable and is in a range from about 10:90 to about 35:65; and
the radiopaque group is a residue of compound a highly iodinated benzoic acid containing compound having at least 4 iodine groups.

20. The cross-linked reaction product of claim 18, wherein crosslinked reaction product exhibits improved cross-linking kinetics, in vivo gel persistence, improved radiopacity, or any combination thereof.
